(19) United States
(12) Patent Application Publication
(10) Pub. No.: US 2023/0233847 A1
(43) Pub. Date: Jul. 27, 2023

(54) PERSONAL PROTECTIVE EQUIPMENT

(71) Applicant: First Step Holdings, LLC, Tucson, AZ (US)

(72) Inventors: Scott N. Sheftel, Tucson, AZ (US); Jeffry B. Skiba, Chandler, AZ (US)

(21) Appl. No.: 17/920,177

(22) PCT Filed: Apr. 20, 2021
(86) PCT No.: PCT/US2021/028238

§ 371 Date: Oct. 20, 2022

Related U.S. Application Data
Application No. PCT/US2021/028238 is a continuation of application No. 17/110,093, filed on Dec. 2, 2020, now abandoned.
Application No. PCT/US2021/028238 is a continuation of application No. 16/952,938, filed on Nov. 19, 2020, now abandoned.
Application No. PCT/US2021/028238 is a continuation of application No. 17/073,261, filed on Oct. 16, 2020, now abandoned.
Application No. PCT/US2021/028238 is a continuation of application No. 16/941,371, filed on Jul. 28, 2020, now abandoned.
(60) Provisional application No. 63/012,262, filed on Apr. 20, 2020.

(51) Int. Cl. A61N 1/18; A61N 1/04; A61N 7/00; A41D 31/30; A41D 13/11; A61L 9/16; A62B 23/02; B01D 39/08; B01D 53/32; D06M 11/83

(52) U.S. Cl. CPC A61N 1/18; A41D 13/1192; A41D 31/30; A61L 9/16; A61N 1/0476; A61N 1/0484; A61N 7/00; A62B 23/025; B01D 39/086; B01D 53/323; B01D 53/326; D06M 11/83; A61N 1/36014

(57) ABSTRACT

The present invention relates to anti-microbial, antiviral, fibers, and fabrics and to devices made from said fabrics. The invention has particular utility in connection with personal protective equipment (PPE’s) such as surgical masks and respirators, and will be described in connection with such utilities, although other utilities are contemplated, including, for example for forming air handling filters for people movers, i.e., automobiles, trucks, buses, trains, ships and planes, as well as for forming filters for air handling equipment for buildings.

The present invention relates to anti-microbial, antiviral, fibers, and fabrics and to devices made from said fabrics. The invention has particular utility in connection with personal protective equipment (PPE’s) such as surgical masks and respirators, and will be described in connection with such utilities, although other utilities are contemplated, including, for example for forming air handling filters for people movers, i.e., automobiles, trucks, buses, trains, ships and planes, as well as for forming filters for air handling equipment for buildings.

Surgical masks and respirators mitigate the spread of infectious diseases including, but not limited to the common cold, influenza, SARS, H1N1 Swine Flu, and most recently. COVID-19, also known as “coronavirus.” Surgical masks and respirators and masks are designed to reduce the spread of airborne illnesses by providing a physical filter between facial regions of the wearer’s and the wearer’s ambient environment. Surgical masks are less effective than respirators, which provide a tighter seal around the nose and mouth and provide better air filtration. Surgical masks are also less effective than respirators at reducing the spread of viral or other microbial infections via aerosolized particles, making them a risky form of personal protective equipment for health care providers dealing with influenza, COVID-19. and other infectious microbes. Effective prevention of the spread of airborne illnesses is particularly important for healthcare providers and first responders, who frequently come into contact with infected and non-infected patients.

Common masks used by non-medical professionals, i.e., paper or cloth masks, which are only partially effective at reducing the spread of viral or other pathogen infection through inhalation and exhalation. Paper masks are not regulated and while they have been established as more effective than no barrier, their efficacy is variable, with only 30-50% barrier efficacy in some instances, which may provide users a false sense of security leading them to acquire or spread infection. Unregulated paper masks typically are not multi-ply and do not provide respiratory protection. Paper masks are mainly useful at preventing the user from touching the area around their nose and mouth, and are only marginally useful for preventing a patient from contracting infection or from preventing an infected patient from spreading infection. Nevertheless, for aerosolized virus, they offer better air filtration of viral pathogens than no mask at all. During times of pandemic when personal protective gear must be rationed due to high demand, the need for a continuous supply of replacement masks places financial and medical strains on the health care system.

Surgical masks are loose-fitting and disposable, and often wrap around the ears to cover just the nose and mouth. Most surgical masks are multi-ply, providing better filtration than paper and cloth or homemade masks. Some surgical masks have an additional face shield. Surgical masks are regulated, unlike cloth or paper masks, and reduce the risk of contracting or spreading infection by filtering out a degree of small particles such as viruses. Surgical masks are used by doctors, surgeons, and dentists during medical procedures for maintaining a sterile procedure and preventing fluid transmission between healthcare providers and patients. However, there is still risk of infection transfer as surgical masks have been shown to have reduced efficacy reportedly around 80% of particles for air filtration, which aids in preventing the spread of viral pathogens either via exhalation or inhalation. Surgical masks also serve as a barrier to liquid splashes including saliva. However, surgical masks do not cover eyes to prevent ocular transmission of aerosolized pathogens. Surgical masks are frequently worn in East Asian culture, including in Japan and Taiwan, to reduce the risk of spreading infection and as a sign of social responsibility to alert others that the person may be infectious.

Respirators provide further protection against bilateral spread of infection preventing the wearer from being exposed to infection and preventing an infected person from exposing others. The most common respirators are disposable N95-NI00 respirator masks. Respirators under optimal circumstances are designed to be tight-fitting around the nose and mouth area and filter out small particles including virus. Respirators, when perfectly fitting, may filter out 95%-100% of airborne particles as small as 0.3 microns. Respirators, in conjunction with other personal protective equipment are highly effective at reducing the spread of viral and bacterial pathogens and commonly used by research and medical professionals. However, there are inherent limitations of the effectiveness of the masks when used by wearers who have facial hair, who experience perspiration on the face that limits the occlusive fit of the mask, or who’s facial shape does not allow a perfect or secure fit. Furthermore, in the case of COVID 19 the virus is extremely tiny (less than 0.2 microns) making an adjunctive means of anti-microbial activity in a mask more important. Additionally, as mask efficiency is increased, e.g., through use of additional or thicker or tighter layers, the pressure required to pull air through the mask is increased. Pressure also increases as the layers become loaded with particles. Filter designs which include tortuous pathways which slow particle velocity and/or trapping of particles also increase pressure. Improving the antimicrobial milieu of the breathing chamber in a mask may help overcome the limitations of an imperfect respirator and improve the antimicrobial environment of a face shield.

Furthermore, some respirators, including N95 masks, are disposable, in order to eliminate the opportunity for daily contamination when exposed to infected persons or patients, and to avoid the potential spread of infection between health care providers with each other or spreading infection between patients. It is assumed respirators become contaminated when doctors come into contact with infected patients, particularly for aerosolized types of infection-or when worn by an infected person . Unfortunately, in the event of a shortage of personal protective equipment, including face masks, healthcare providers and first responders are forced to reuse face masks, increasing the likelihood of becoming infected themselves and of spreading infection to others. Conventional respirators when worn properly by a person infected with viral, bacterial, or fungal pathogens decrease the spread of their droplets by keeping them trapped in the face cup. However, these devices do nothing to decrease the level of infectious pathogens already present on skin or viral reservoirs in an infected patient’s nasal or oral cavities. In fact, face masks on an infected person in some instances may actually create the moist environment that could increase viral replication.

Furthermore, face masks and respirator N-95-100 masks which are disposable and easily contaminated, require large volumes of equipment to maintain supply in times of pandemic, causing shortages and limiting public access to these items in order to necessarily maintain the health and protection of health care providers and other essential workers.

While facial coverings, i.e., facemasks or facial mask inserts or replaceable cartridges or PPE masks, fabrics, or barriers including gloves, are considered by health experts to be a first line of defense against transmission of various diseases, and the use of face masks has become a critical tool in fighting spread of disease during the current Covid-19 pandemic, for many people, the use of face masks may result in skin irritation or allergies. This can be a significant problem, particularly for healthcare and essential workers who must wear face masks all day.

One reason irritation occurs is that the face masks do not allow free airflow to the face, since face masks are designed to be worn closely fitting to the face. Thus, when the wearer breathes, moisture or oils may accumulate and become trapped on the face. The resulting dark, warm environment can cause skin issues such as acne or “mask rash”. In addition, face masks and other facial coverings can irritate the skin simply by rubbing against it, or by exposing the skin to allergens. Also, the type of material and its contact with the skin may have a negative effect resulting secondary irritation or contact dermatitis.

In our earlier U.S. Pat. 9,192,761 and U.S. Pat. 9,707,172, we describe devices for treating hyperhidrosis. Hyperhidrosis is a medical condition in which patients experience excessive sweating. Excessive sweating can lead to a patient’s physical and societal discomfort, and may also lead to skin irritation and other skin problems. More particularly, the device described in our aforesaid prior US Patents, comprises a fabric having zinc particles disposed on at least a portion of the fabric, wherein the fabric is configured to contact a body surface such that the zinc particles come in contact with a skin surface, whereby to create a zinc-oxide battery which effectively treats hyperhidrosis. Also, as reported in our U.S. Publication No. US 2019/016190, our prior patented devices also may be used to treat a variety of other conditions suffered by both humans and animals, including for example, neuropathic pain (including peripheral artery disease and neuropathy, surgical rehabilitation including joint surgery rehabilitation, surgery convalescence including joint surgery rehabilitation and soft tissue healing; physical therapy including muscle and tendon healing and stroke rehabilitation. Our aforesaid patented devices also are said to enhance athletic performance, endurance and to promote faster recovery after exertion along with less muscle discomfort and fatigue.

As described in our aforesaid patents and pending applications the particles of zinc, zinc oxides or zinc salts are carried on a surface of a fabric as a plurality of dots or lines in a specific pattern that positions the zinc reservoirs in discrete locations, each location separated by a distance. When this zinc-carrying fabric is placed with the zinc particles in contact with the skin of a person or animal, the zinc particles separation and configuration couples with oxygen and moisture at the skin surface to create a zinc-oxygen battery which produces an electric current at the skin.

The mechanism is as follows: The zinc-carrying fabric pattern acts as a half-cell anode and the oxygen partially supplied by circulation at the skin surface acts as a half-cell cathode physically separated to allow electric fields to exist. The human or animal’s body contributes moisture, which completes the circuit to allow current to flow as a function of impedence within the tissues. The completed circuit creates a redox reaction with oxidation of the zinc and reduction of the oxygen (2Zn+O₂→2ZnO). The oxygen is ambient or replenished with the circulating blood oxygen (partial pressure of oxygen diffusing through the skin) at the skin’s surface.

Microcurrent stimulation is a known phenomena in the range of millionths of an ampere. Humans and other animals have inherent electrical (microcurrent) properties that drive and maintain their bodies. Cells communicate with one another via complex neuro pathways generated and maintained by biochemical reactions that create electrical activity and these endogenous point charges, electric fields and electric currents all have a function in cell signaling such as migration patterns, expression of reactive oxygen species, and regulation of gene expressions. The body generates electrical fields in vital organs such as the heart and brain that are easily measured with instruments such as EEG (electroencephalogram), and EKG (electrocardiogram). The electrophysiology of the human body indicates that cells and organs possess an electrical nature. Studies of electric field and microcurrent stimulation have been well documented for decades. The effect on the human body is evident both clinically and on a cellular level. Physiologic studies document increased capillary density, enhanced blood flow and tissue oxygenation, as well as an enhanced cellular response with increased protein synthesis, amino acid transport and increased ATP (mitochondrial energy) synthesis. In addition to amplifying critical cellular functions within the cell, microcurrent also may increase local cellular absorption of nutrients and facilitates waste elimination, a critical component of muscle performance and recovery.

While some doses of electricity stimulate cellular activity, specific doses can suppress or inhibit cellular function. An example of inhibitory activity is seen with the effect of electrical current on sweat production and bacterial growth. The efficacy of applying external electrical current to the skin for control of excessive sweating (hyperhidrosis) is historically well documented. This concept is the basis for hyperhidrosis treatments utilizing external battery devices such as marketed under the name Drionics, available from General Medical Company. In addition to reducing sweat gland activity, electrical current inhibits the activity of bacteria and fungi, the organisms responsible for foot odor and athletes foot.

In US 2019/0161910 A1, we describe a method for producing metal particle filled fibers by dispersing metal particles throughout the fibers during fiber production, and to metal particle filled fibers produced thereby. Preferably, the metal particles include zinc particles, zinc oxide particles, or zinc salt particles, having a particle sized range of 1 micron - 200 microns. The metal filled fibers may then be used to form fabric devices for treating hyperhidrosis and other conditions such as neuropathic pain including peripheral artery disease and neuropathy; surgical rehabilitation and surgical convalescence including joint surgery, rehabilitation and soft tissue healing; and physical therapy including muscle and tendon headlong and stroke rehabilitation by applying directly onto a skin surface of a patient in need of such treatment, a device comprising a fabric or substrate containing discrete patterns of elemental zinc particles arranged so that the fabric or substrate in contact with the skin of the wearer forms a plurality of half-cells of an air-zinc battery, whereby to produce an ion exchange with the skin of the patient. Zinc, zinc oxide or zinc salt particles against the skin also will result in secondary reactions to form zinc complexes beneficial to the host. The ability to deliver topical zinc to the surface of the skin can have beneficial effects provided the zinc particles are in the correct physical arrangement.

Additionally, the therapeutic value of metals and metal salts such as zinc, zinc oxide and zinc salt in cosmetic and medicinal ointments and creams, i.e., for treating a variety of skin conditions is well documented in the art. However, one of the limitations of creams or ointments is that they require a carrier gel or petrolatum, and these carriers create barriers on the skin, potentially trapping microbes beneath the barriers.

We have now found that fibers containing zinc particles, particularly discrete patterns of nano size zinc particles for forming fabrics incorporated into personal protective equipment such as masks, provide an anti-microbial kill rate in excess of 99% when in close proximity to the skin of a human or other animal. This is unexpected since micron size particles of zinc as disclosed in our aforesaid US Published application and other a prior art required that the zinc particles needed to be in contact with the skin of the wearer to generate an electrical field to have any antimicrobial effect. In accordance with one aspect of our invention we have now found that by employing nano size zinc particles electrical fields are formed in the fabric even in areas not in direct contact with the skin of the wearer. All that is necessary is that the fabric contact the skin of the wearer at some point. While not wishing to be bound by theory, it is believed that as the zinc particles approach < 1000 nanometers in size, quantum effects begin to apply substantially increasing surface energies and field effects which bridge areas of the fabrics and which result in kill rates of microbes not seen with larger size particles. More particularly, we have found that fibers containing nano-size particles of zinc, preferably 1 to 1,000 nanometers, even more preferably 1 to 100 nanometers sized particles have a kill rate in excess of 99% against various microbes or pathogens, including but not limited to viruses causing the common cold, influenza, SARS, H1N1(swine flu) and COVID-19, as well as bacteria, algae, fungi, molds, yeasts, etc. This is unexpected since larger size zinc particles incorporated into fibers do not provide similar anti-microbial properties.

Preferably the fiber material a comprises thermoplastic polymer, preferably polyethylene, although polypropylene, various thermoplastic polymer materials and natural fibers may be used. Preferably the zinc-nano particles are incorporated into the polymer fibers on formation of the fiber. However, the zinc nano particles also could be applied to the surface of the fibers using binders, or heated nano particles may be sprayed directly onto the surface of the fibers. The nano particles also may be wiped directly onto the surface of the fiber and into interstities in the fiber surface, for example by pulling the fibers through a “bath” of nano particles, or wiping the nano particles onto the fibers. The nano particles also may be printed into the surface of the fibers. In order to strengthen the fiber, carbon nanotubes may included in the fiber during fiber formation.

In another aspect of our invention, we now have found that zinc loaded fabrics made having spaced particles of elemental zinc, zinc oxide and/or zinc salt and certain other metals, such as copper, silver and magnesium, and oxides and salts thereof advantageously can be used to form filter media which can act as a barrier to a variety of airborne pathogens and viruses, i.e., as filter media in PPE Masks, air cleaners and/or filters HVAC units in hospitals and nursing homes, residential and commercial building applications, as well as transportation applications - anywhere air must be cleaned of airborne pathogens or viruses.

An essential element in the design considerations of an air filter media is to minimize static pressure drop across the media. We have found we can provide zinc loaded fabrics that exhibit minimal static pressure drop without compromising zinc oxygen battery formation or efficiency. This feature is especially useful to reduce heat or humidity buildup inside of a PPE mask. The zinc oxide battery also kills COVID and other pathogens, such as H1N1, SARS, MERS and pneumonia, while keeping skin healthy.

In one aspect of our invention there is provided a method of inactivating or killing airborne bacteria, fungi, molds and viruses comprising passing the air through a filter material formed of a fabric having particles of zinc disposed in discrete physically isolated locations, wherein the particles of zinc and exhaled oxygen form half cells of a battery. There is moisture in exhaled air that completes the circuit to create an electrical field that inactivates or kills airborne bacterial, fungi, molds and viruses passing through the filter material.

In one embodiment, the fibers are spatially separated within the fabric to set up an electric field as determined by the weave pattern or the knit pattern.

In another embodiment, the fiber surface area of the fabric can be increased by flocking, felting and/or terrying which would allow designs to slow down passage of airborne particles and expose more of the active fibers to the airborne pathogens.

In another and preferred embodiment, the filter material is formed into a face mask as either a single layer or as part of a composite which could include absorbents, water proofed, or other performance fabrics that could increase overall performance.

In yet another embodiment the fabric is formed of threads or filaments having particles of zinc, and threads or filaments having particles of copper, silver or magnesium, woven or with a neutral insulating fiber or thread as part of the weave, knot or non-woven process. The neutral thread is present to separate the active fibers from each other to form electric fields which would not exist if the fabric was coated completely.

In a further embodiment wherein the filter material is a fabric formed of fibers or filaments having particles of zinc, zinc oxide and/or zinc salt, and fibers or filaments having particles of copper, silver or magnesium, copper, silver or magnesium oxide, or copper, silver or magnesium salt wherein the filaments are woven, knitted or thermally fused, and separated at least in part from one another. These fibers may be used to form a fabric or mesh with batteries of several different cell types creating areas of high and low field strengths, of batteries that would exhaust with time while others continue to provide energy. In this embodiment, a high electric field could be designed to last for a period of time while a second electric field construct could initiate when the first dies away, and so forth. Batteries are defined by field strength and capacity. We can control the field strength with the half cell, and the capacity with the amount of zinc, copper, etc within the cell. In a biologic environment we also can use bioresorbable polymers to slowly degrade exposing more of the cell for reaction. An example would be a conical shaped reservoir where the large area of the cone would provide higher capacity and as the cone gets smaller, the field shape changes and the capacity is reduced until more cone is exposed.

In another embodiment oxygen carried by the air passing through the filter material reacts with the zinc to form a half-cell.

In another embodiment oxygen carried by the exhaled air passing through the filter material reacts with the zinc to form a half-cell.

In still another embodiment supplemental oxygen is provided from an external oxygen source selected from hyperbaric oxygen, hydrogen peroxide, or an oxygen concentrator or other.

The present disclosure also provides a filter capable of inactivating or killing airborne bacteria, fungi, molds and viruses, said filter being formed of a material comprising a filter material formed of a fabric having particles of zinc disposed in discrete physically isolated locations, wherein the particles of zinc and oxygen from the air form half cells of a battery whereby to create an electrical field that inactivates or kills airborne bacterial, fungi, molds and viruses passing through the filter material.

In one embodiment, the fibers are spatially separated within the fabric to set up an electric field as determined by the weave pattern or the knit pattern.

In another embodiment the fiber surface area of the fabric is increased by sanding, flocking, felting and/or terrying.

In yet another and preferred embodiment the filter is in the form of a face mask.

In another embodiment, the fabric is formed of threads or filaments having particles of zinc, and threads or filaments having particles of copper, silver or magnesium, copper, silver or magnesium oxide, or copper, silver or magnesium salt, woven or with a neutral insulating fiber or thread as part of the weave, knot or non-woven process.

In still another embodiment, the filter material is a fabric formed of fibers or filaments having particles of zinc, zinc oxide and/or zinc salt, and fibers or filaments having particles of copper, silver or magnesium, copper, silver or magnesium oxide, or copper, silver or magnesium salt wherein the filaments are woven, knitted or thermally fused, and separated at least in part from one another.

In another embodiment the filter is in the form of an air filter configured for use in a people mover.

In still another embodiment the filter is in the form of an air filter for use in air handling equipment for buildings.

An additional feature of our zinc oxygen battery technology is that in contact with the skin, it is highly beneficial and increases the Collagen 1 Collagen 3 ratio, and aids in skin tissue health. Other technologies used to minimize mask rash such as bleach loaded fabrics or copper loaded fabrics do not possess this feature.

Further features and advantages of the instant invention will be seen from the following detailed description taken in conjunction with the accompanying drawings, wherein

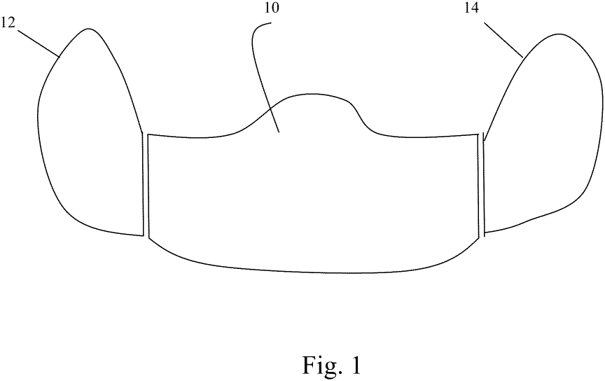

FIG. 1 is a plan view of a filter face mask made in accordance with a preferred embodiment of our invention;

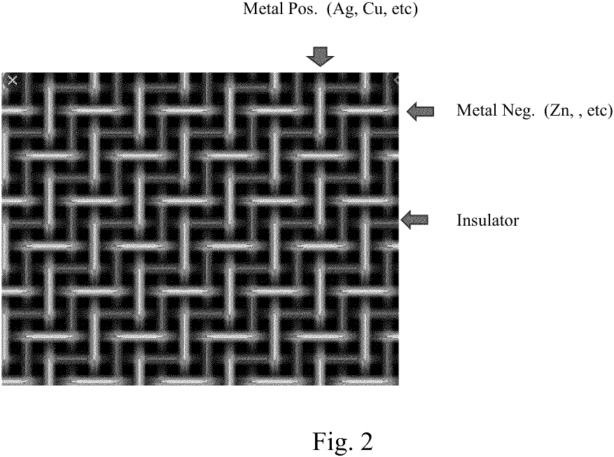

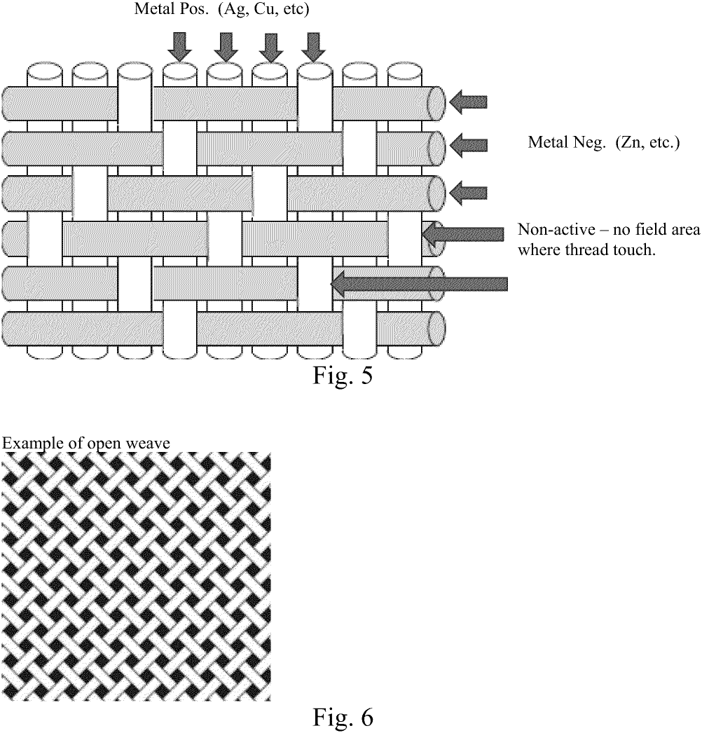

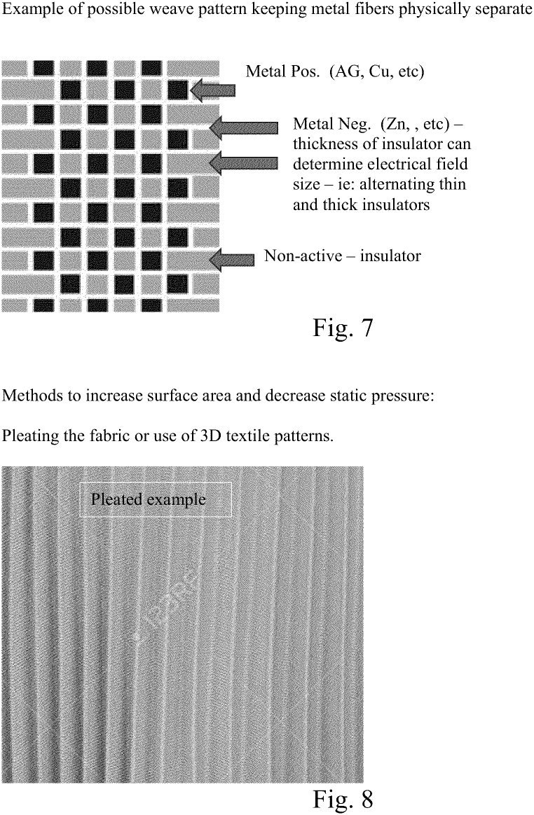

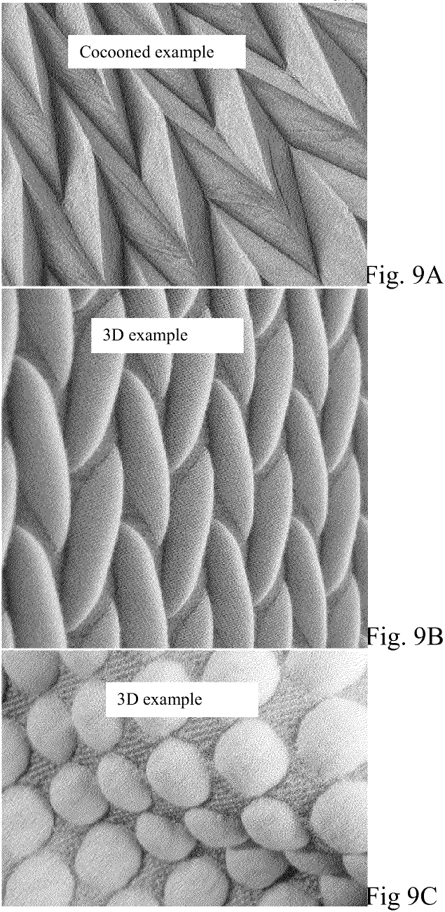

FIGS. 2-8 are two dimensional views of fabrics useful in forming filters in accordance with the instant invention;

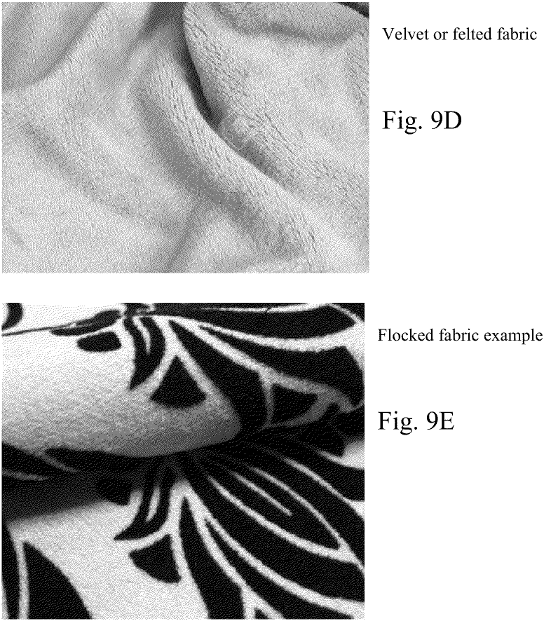

FIGS. 9A-9E are three dimensional views of fabrics useful in forming filters in accordance with the instant invention;

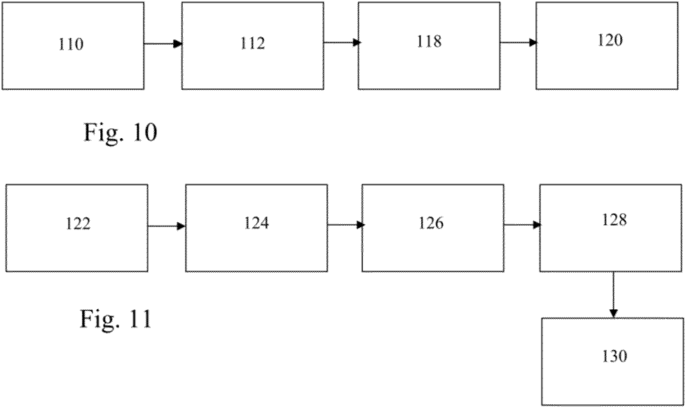

FIG. 10 is a flow diagram showing a preferred method of forming nano particle size metal particles coated fibers in accordance with the present invention;

FIGS. 11-14 are views of alternative methods for forming nano particle size metal particles coated fibers in accordance with the present invention;

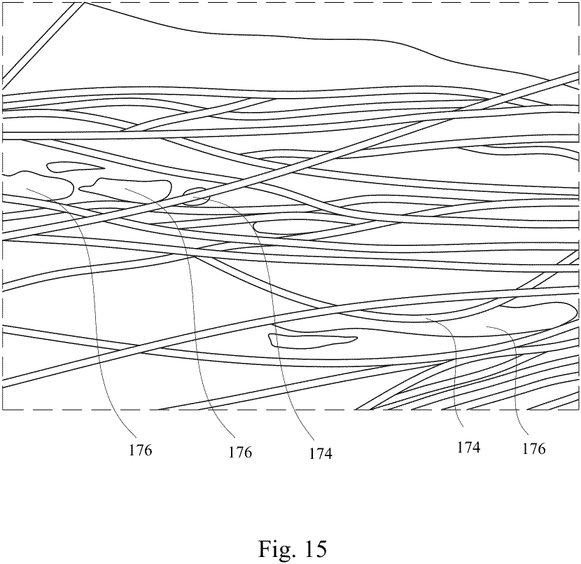

FIG. 15 is a side elevational view of monofilaments fiber made accordance with the present invention;

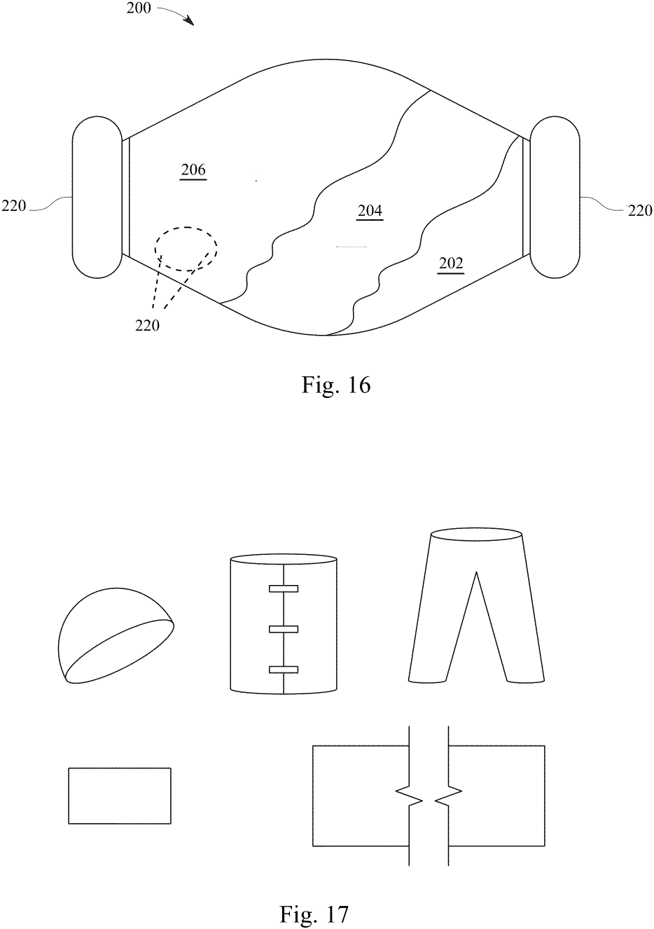

FIG. 16 is a plan view of a surgical mask formed in accordance with the present invention;

FIG. 17 is a plain view showing various articles of personal protective equipment made in accordance with the present invention;

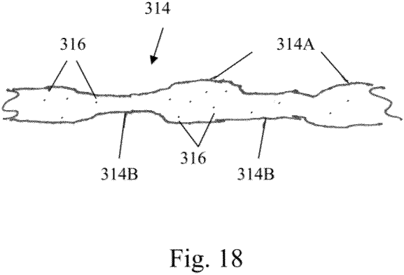

FIG. 18 is a side elevational view of a metal particle filled fiber made in accordance with the present invention;

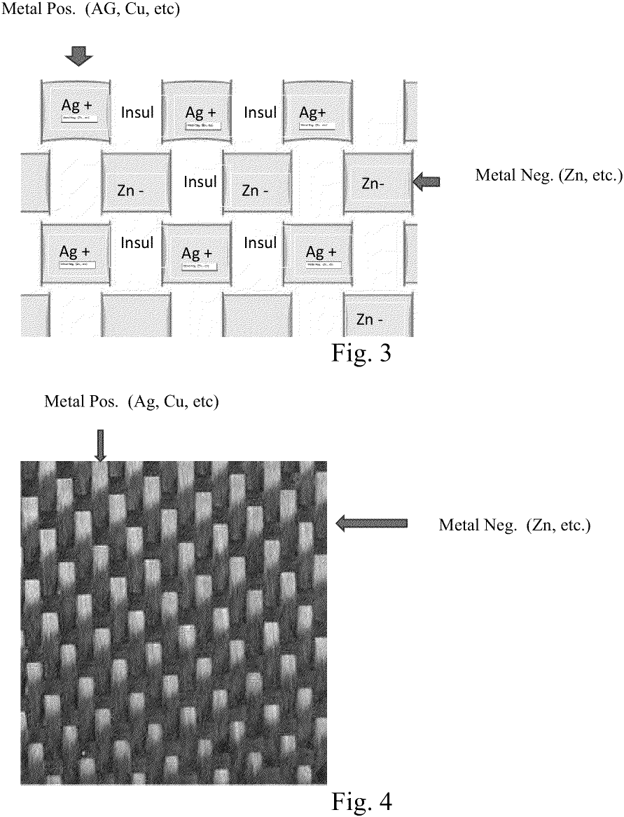

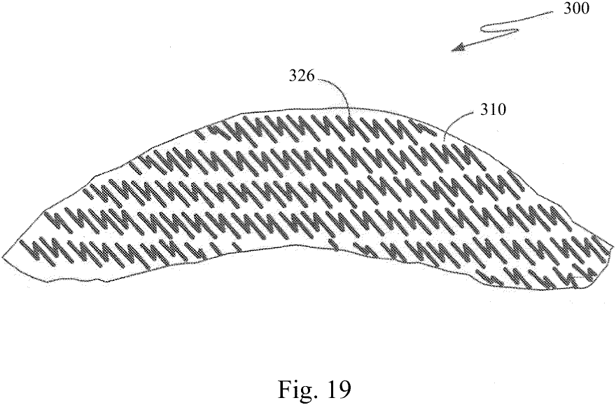

FIG. 19 is a top plan view of a fabric made from a monofilaments fiber of FIG. 3 in accordance with the present invention; and

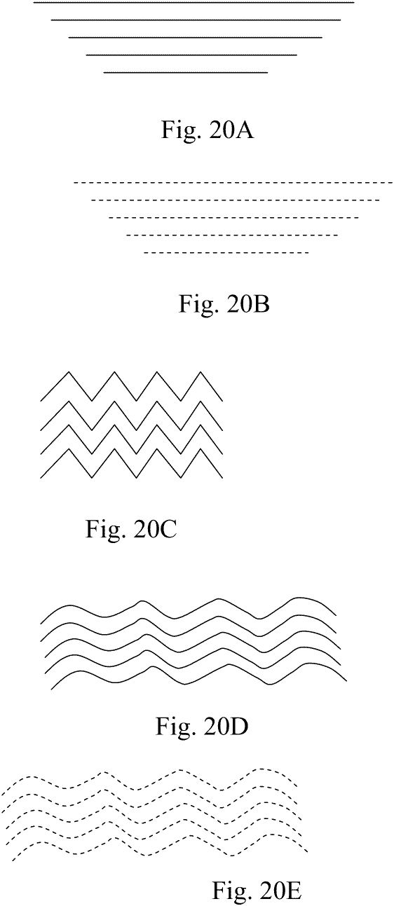

FIGS. 20A-20E illustrates patterns of metal deposition on fabric used for making articles of clothing in accordance with the present invention.

As used herein the term “microbe” or “pathogen”, which are used interchangeably, may include bacteria, algae, fungi, molds, yeasts, and viruses including but not limited to the common cold, influenza, SARS, H1N1, Swine Flu and COVID-19 commonly know as “Coronavirus”.

“Personal protective equipment” or PPE may include masks, scrubs, respirators, caps and other headgear such as face shields, and other types of clothing as well as sheets, pillowcases, and the like.

“Metal particles” may include elemental zinc particles and oxides and salts thereof.

“Fibers” include natural and artificial fibers, preferably thermoplastic and thermosetting fiber materials more preferably, polyethylene.

And “metal filled fibers” means fibers, having metal particles carried on or within the fibers, and in which at least some of the metal particles are at least in part exposed to air.

A zinc oxygen battery produces between 0.1 and 1 Volt providing an electric field by design, keeping positive and negative poles slightly separated and not shorted out. This physical separation is essential and creates the unique nature of our electrically active fabric. The amount of fiber, the concentration of the metal on the surface, the particle size of the metal power, the blend of neutral/active fiber, how the fiber is drawn through thermal spinning, the denier or weight of the fiber and the construction of a thread or yarn all may contribute to the battery efficiency and may affect static pressure drop when the fibers are formed into a fabric and used as a filter. Also, our fibers having particles of zinc, zinc oxide or zinc salt, or particles of copper, silver or magnesium could be blown into a melt or non-woven fabric used for form filter media.

Also, physical characteristics of the fiber, weight, size, weave, and other physical characteristic may significantly affect filter efficiency, filter life and static pressure drop. Balancing these characteristics and preparation is critical to forming a useful filter. Zinc and certain other metals may be used to create cells capable of providing voltages; however, it is the creation of electric fields at a small scale within a filter fabric that generates a field at a scale where microbes, bacteria, viruses, and other pathogens will be affected. The use of weaving or knitting allows the design of a variety of patterns that can be mass produced and eventually converted into filter media with active electrical activity. For example, in addition to zinc, an additional dissimilar metal, preferably, copper, silver or magnesium or an oxide or salt thereof, can be added to the fabric to initiate a galvanic cell between the dissimilar fibers physically separated to create an electric field. Field strengths are a function of the half-cell potential of the metals. For example, Zn is -0.75 eV and Ag is +0.76 eV for a theoretical voltage maximum of 1.5 Volts. In a Zn/Cu cell construct the Cu would be at 1.10 volts.

In one embodiment of our disclosure, the fiber can be used to create a woven pattern wherein there is physical separation of metal infused fibers, threads or yarns. This is done using a weave pattern where three fibers or thread are used. One fiber is a positively charged, one fiber is negatively charged and one fiber is a neutral or insulating fiber or thread. In the weaving process the individual active threads can be physically separated by the insulating thread at a distance determined by the thickness and number of insulating threads between the active threads. Active threads can be single or multiple fibers or threads/yarns of a predetermined thickness that will eventually contribute to the weight and feel of the finished fabric. The weave pattern can be loosely woven or tightly woven depending upon the desired electrical output, while balancing static pressure drop. In an embodiment where the woven fabric is to be used for filtration masks the thickness of the fabric, the space between the fibers or threads, the weight of the fabric all contribute to static pressure or the force required to pass air through the woven fabric. A tight weave increases static pressure and a loose weave reduces it.

A woven pattern can be selected that uses a neutral, insulating thread to physically separate the two dissimilar metal active threads. In an alternative embodiment, the two active threads may come into contact with each other at intersections. However, at those intersections, the active electrical field will be lost.

Other techniques such as sanding, felting, terrying, and flocking may be used to increase the available surface area of the active fibers and therefore the amount of active barrier in the filter media. Through these methods, the surface area per unit is increased by lifting the fibers away from the base fabric.

Referring to FIG. 1, a face mask made in accordance with the incident disclosure comprises a main body 10 shaped to fit over the nose and mouth of the wearer. Straps 12, 14 are affixed to the respective distal end of body 10 for fastening the face mask over the ears or behind the head of the wearer. The face mask is similar to take the conventional face mask, except the mask body is formed of a fabric today having elemental zinc particles infused into the fibers of the fabric exposed in part on a surface of the fabric so as to come into contact with the skin of the wearer. The fabric is formed by weaving filaments containing zinc particles spaced from one another, following the teachings of our aforesaid U.S. Pat. Nos. 9,192,761 and 9,707,172, and our published U.S. Application Serial No. 2019/016190 and our PCT Published Application WO 2019/241074, the contents of which are incorporated herein by reference, with filaments optionally containing particles of copper, silver or magnesium spaced from one another, in a basket weave as shown in FIGS. 2 and 3. Preferably the metal particles are zinc particles and have an average particle size of between 1 and 100 nanometers, more preferably 1 to 10 microns, and even more preferably about 5 microns. The metal particles may be printed or bound on a substrate fabric, or extruded or melt spun at the time of fiber formation as taught by our aforesaid patents and pending applications. The amount of zinc and the surface area of the zinc or other metal used is a function of particle size and availability to create the battery.

Preferably, but not necessarily, the fabric comprises a woven textile, although the fabric may be a non-woven textile, a fibrous mesh, a non-fibrous mesh, which may include an adhesive coated textile or fabric, mesh or the like.

As taught in our aforesaid ‘761 and ‘172 patents or as described in our pending applications, the metal particles are discontinuously and substantially uniformly distributed on the surface of the fabric, in imaginary spaced lines or lines of dots, across the surface area of the fabric, at least in part. Typically, the lines or lines of dots are evenly spaced at spacings from 0.1 to 3 mm, preferably 0.2 to 2 mm, more preferably 0.3 to 1.5 mm, most preferably 0.5 to 1.0 mm. The concentration of metal such as zinc in the binder or in the extruded fibers that forms the lines or dots determines the amount of metal available for the “battery”. Preferred concentration is 30% of the surface area of the fabric; however, the concentration of zinc may range from about 1% to about 99%. A mixture of binder and zinc metal may be formed as a paste and applied by silk screening e.g., as described in our aforesaid ‘761 and ‘172 patents. A 30% by weight zinc-to-binder is preferred for this. The line or dot width and length also determines the amount of metal in the deposition since the wider and longer the line, the more metal is available. Preferred line dots width is 1 mm width but width can vary from 0.1 mm up to 5 mm width. Since the deposition is on a fabric or carried in the adhesive, the amount of binder/metal applied also can be varied. In certain embodiments, the fabric being coated can be coated twice or more times over the same pattern whereupon the thickness of the deposition can be increased as desired. In certain embodiments, the metal deposition area patterns cover from about 10% to about 90% of the surface area of the fabric. In other embodiments, the metal deposition areas cover from about 20% to about 80%, from about 15% to about 75%, from about 25% to about 50%, or from about 30% to about 40% of the surface area of the fabric or anywhere in between. Although the drawing figures show the plurality of metal deposition areas substantially uniformly distributed on the surface of the fabric, in other embodiments, the plurality of metal deposition areas may be randomly distributed on the surface of the fabric. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes as shown, for example, straight continuous lines; straight broken lines; continuous saw-shaped; continuous wavy lines; broken wavy lines, etc, as described in our aforesaid ‘761 and ‘172 patents and our pending applications. The actual shape of the lines is not important. Preferably, but not necessarily, the lines are approximately equal in thickness and are evenly spaced.

In a preferred embodiment the metal particles previously formed by grinding or precipitated out of suspension, and having an average particle size between 1 and 100 nanometers, more preferably 1-10 microns, even more preferably about 5 microns are mixed with a thermal plastic material such as polyethylene in a heated mixing vat 30 to melt the material, and the mixture extruded or melt spun at spinning station 32 to form fibers, having metal particles contained therein. The metals containing fibers may then be cabled or twisted at a cabling station, and woven at a weaving or knitting station into a sheet or cloth. The resulting metal particle impregnated sheet or cloth is cut to size and formed into a mask.

Other weave patterns are possible as illustrated in FIGS. 4-8, or 9A-9C. All that is required is that the at least some of the fibers or filaments making up the fabric include particles of elemental zinc, zinc oxide or zinc salt and another elemental metal, metal oxide or metal salt, e.g., of copper, silver or magnesium are separated within the fabric so as to set up an electric field as described in our aforesaid patents and pending application.

Various changes can be made in the above disclosure without departing from the spirit and scope of our disclosure. For example, the efficiency of the via media as a filter media may be increased by increasing the loading of zinc, zinc oxide or zinc salt or and/or other elemental metal, metal oxide or metal salt; employing finer particles; or by modifying the surface of the fibers of for example, by sanding, felting, flocking or terrying to create an increased surface area/volume, or pleating the fabric so as effectively to increase the surface area of the fiber, by lifting the fiber in part from as the base fabric, as illustrated in FIGS. 9D-9E.

When used as a mask, the metal particle fiber matrix interacts with exhaled moisture and oxygen, or moisture and oxygen from the wearer’s skin surface, and/or ambient moisture and oxygen to generate a microcurrent. An electric field is created without an external battery source, which destrous virulent microbes or pathogens including Coronavirus. The filter material of the present disclosure also has several other advantageous effects.

Zinc is a co-factor and is essential to bodily functions. One of its roles is to lessen the formation of damaging free radicals and protects skin’s lipids (fats) and fibroblasts—the cells that make collagen, one’s skin’s support structure—when skin is exposed to UV light, pollution and other skin-agers. It helps heal and rejuvenate skin. When there is an insult or trauma to the skin. Zinc is essential to the heating process and health of the body. Zinc also is essential to the metabolic process’s and health of the body. Zinc lessens the formation of damaging free radicals and protects skin’s lipids (fats) and fibroblasts—the cells that make collagen, one’s skin’s support structure—when skin is exposed to UV light, pollution and other skin-agers. It helps heal and rejuvenate skin. When you cut yourself, zinc goes to work.

Oxygen has a unique effect on the skin because it is important for cellular function and metabolic process. In the presence of Oxygen, the permeability of the skin barrier is enhanced and the skin is more receptive to exogenous stimuli. Also, oxygen has a unique effect on the skin because it opens up our pores, increasing their absorption power. After being exposed to oxygen, the skin starts breathing again and all treatments applied thereafter produce even better results.

Additionally, microcurrents send low-level electrical currents into the wearer’s skin that are nearly identical to the body’s own natural electrical frequencies, i.e., similar to the effect when physical therapist places electrodes on target areas of the body, or, like getting a microcurrent facial.

Microcurrents also stimulate the wearer’s facial muscles for a natural lift, i.e., similar to microcurrentfacials which tighten and smooth the muscles and connective tissues in the face by increasing cellular activity, and have been shown to reduce wrinkles, mostly around the forehead area. And, microcurrents also work at the cellular level to literally recharge the wearer’s skin back to a more youthful state, and results in increased levels of ATP which speeds cellular metabolism, stimulates protein synthesis, promotes detoxification and reconstitutes collagen and elastin.

The present invention also provides a method forming nanosized metal particle filled fibers suitable for weaving or knitting into a fabric for use in forming personal protective equipment. More particularly, the present invention in one aspect provides a method for producing nanosized metal particle containing fibers that are capable of forming metal-air electrochemical cells, capable of releasing ions when adjacent or in contact with a wearer’s skin or moisture.

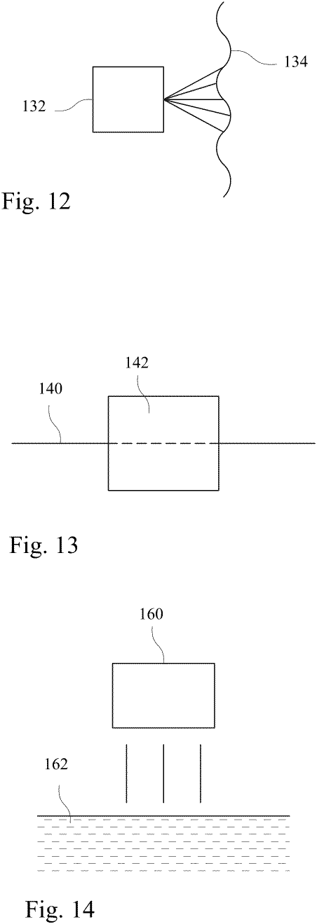

Referring to FIG. 10, according to another embodiment of our invention, metal particles, typically metallic zinc particles which may be previously formed by grinding or precipitated out of suspension, and having an average particle size between 1 and 1,000 nanometers, more preferably 1 to 500 nanometers, even more preferably 1 to 100 nanometers are mixed with a thermoplastic material such as polyethylene in a heated mixing vat 110 to melt the thermoplastic material, and the mixture bump extruded or melt spun at spinning station 112 to form fibers 114, having nanometer size metal particles 116 (see FIG. 12). Polyethylene is the polymer of choice for releasing of electrons from the metal. The porosity of the fiber also is believed to play a part. Polyacrylic or polyester fibers also may be used; however polyacrylic or polyester fibers result is a slower ion release. The nanometer sized metal particles filled fibers may then be cabled or twisted at a cabling station 118, and woven at a weaving or knitting station 120, or laid in a non-woven manner, into a fabric in which the zinc nano particles are separated in discrete patterns or lines as described in our aforesaid US Published Application, and in our earlier U.S. Pat. Nos. 9,192,761 and 9,707,172, the contents of which are incorporated herein by reference, which is then used to form personal protective equipment such as a mask as previously or as described below with reference to FIG. 16 described, or made into a hospital scrub or cap, gown or scrub, or a sheet, pillow case, towels, wipes, etc., as shown in FIG. 17.

Referring to FIG. 11, according to a second embodiment of the invention, nanosized metallic zinc particles having an average particle size between 1 and 1,000 nanometers, preferably 1 to 500 nanometers, even more preferably about 1 to 100 nanometers are mixed with a thermosetting polymer material such as polyester chips in a melting vat 122. The molten mixture is expressed through a spinneret at station 124 to form an elongate thread having metal particles incorporated into the thread with the metal particles exposed at least in part on the surface of the thread. The thread is then cabled or twisted at a cabling station 126, woven into cloth at a weaving station 128, and the cloth with metal-free threads or cables formed into personal protective equipment at step 130.

Referring to FIG. 12 according to yet another embodiment of the invention, nanosized metallic zinc particles having an average particle size between 1 and 1000 nanometers, preferably 1 to 500 nanometers, even more preferably 1 to 100 nanometers, are heated and hot sprayed from a hot sprayer 132 onto preformed fibers or threads 134 whereupon the nano particles adhere to the surface of the fibers or threads. Alternatively, as shown in FIG. 13, a preformed thread 140 are pulled through a vat 142 containing loose mass of nanosized metallic zinc particles of wherein the zinc nano particles key micropores interstices in the fiber surface.

Referring to FIG. 14, and yet another embodiment, metallic zinc particles are printed via printer head 160 onto a surface 162 of a preformed fabric in discontinuous lines as discussed below.

FIG. 15 shows a fabric comprising fibers 174 having zinc particles 176 adhered to the fibers made in accordance with the present disclosure.

FIG. 16 illustrates a mask made in accordance with the present invention. As shown, mask 200 comprises an outer cloth layer 202, a middle cloth layer 204 and an inner cloth layer 206. Outer layer 202 and middle layer 204 are formed of a conventional cloth. Inner layer 206 comprises a fabric having a plurality of spaced metal deposition areas 220. As shown, the plurality of individual metal deposition areas 220 are discontinuous and uniformly distributed on the surface of the fabric 206, in imaginary spaced lines or lines of dots, to cover a substantially consistent percentage of the surface area of the fabric 206. Typically, the lines or lines of dots are evenly spaced at spacings from 0.1 to 3 mm, preferably 0.2 to 2 mm, more preferably 0.3 to 1.5 mm, most preferably 0.5 to 1.0 mm. The concentration of zinc particles in the threads that form the line or deposition determines the amount of zinc available for forming an air-zinc battery as will be described below. Preferred concentration is 30% but the lowest is about 1% and the highest about 50%. In certain embodiments, the metal deposition area patterns 220 cover from about 10% to about 90% of the surface area of the fabric layer 206. In other embodiments, the metal deposition areas 220 cover from about 20% to about 80%, from about 15% to about 75%, from about 25% to about 50%, or from about 30% to about 40% of the surface area of the fabric layer 206. Although FIG. 16 shows the plurality of metal deposition areas 220 substantially uniformly distributed on the surface of the fabric layer 206, in other embodiments, the plurality of metal deposition areas 220 may be randomly distributed on the surface of the fabric layer 206. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes depending on the weave. The actual shape of the lines is not important. Preferably, but not necessarily, the lines are approximately equal in thickness and are evenly spaced.

The mask 200, as illustrated in FIG. 16, comprises a three-layer fabric mask, but alternatively may comprise two, or four or more layers of fabric including the fabric layer containing the metal nano-particles forming the innermost surface or layer of the mask. Alternatively, the metal nano-particles containing a fabric may be formed as filter insert or element on the innermost surface or layer of the mask.

Completing the mask are fasteners such as ear straps or head straps 220 configured to attach the mask to the head of the wearer.

The present invention is unique in that the zinc pattern grid on the tactile layer creates a matrix of individual half-cells (anodes) for ion exchange with the skin of the wearer which effectively kills microbes on or adjacent the skin of the wearer or between the skin and the tactile layer. However, the zinc pattern grid does not have to be in direct contact with the skin of the wearer. One-half cell of electrochemical reaction is the zinc impregnated fabric (the anode), and the other is the skin of the wearer, with the breath of the wearer or moisture from the skin of the wearer, supplying moisture and oxygen (the cathode) completing the circuit for microcurrent production. Alternatively, the oxygen and moisture may be supplied, in part, from ambient air.

There results a Zinc-air battery powered by oxidizing zinc with oxygen from the air. During discharge, zinc particles form a porous anode, which is saturated with an electrolyte, namely moisture from the breath or skin of the wearer or from the air. Oxygen from the air or skin of the wearer reacts at the cathode and forms hydroxyl ions which migrate into the zinc paste and form zinc hydroxide Zn(OH)₂, releasing electrons to travel to the cathode.

The chemical equations for the zinc-air battery formed using Applicants’ zinc-coated masks and ambient oxygen are as follows:
Anode: Zn+4OH⁻ → Zn(OH)₄²⁻+2e⁻(E₀=-1.25 V)
Fluid: Zn(OH)₄²⁻→ZnO+H₂O+2OH⁻
Cathode: ½ O₂+H₂O+2e⁻ →2OH⁻(E₀=0.34 V)

Overall, the zinc oxygen redox chemistry recited immediately hereinabove comprises an overall standard electrode potential of about 1.59 Volts.

There is a certain amount of gas exchange at the skin surface with a partial pressure of oxygen. The oxygen at the skin surface is a product of ambient oxygen in addition to oxygen diffusion from capillary blood flow. In certain embodiments, the zinc in contact with a patient’s skin or breath resulting from wearing, for example, our zinc-containing mask, in combination with moisture from the skin or breath of the wearer and transcutaneous oxygen complete the galvanic circuit described hereinabove.

The chemistry utilized by Applicants’ zinc-coated mask differs from a more conventional galvanic cell. A galvanic cell, or voltaic cell is an electrochemical cell that derives electrical energy from spontaneous redox reactions taking place within the cell. It generally consists of two different metals connected by a salt bridge, or individual half-cells separated by a porous membrane. In contrast, the chemistry of Applicants’ zinc-air battery does not require use of a second metal. Applicants’ device acts as a powerful antimicrobial exhibiting a virus reduction or kill in excess of 99%.

The fabric is configured to contact the skin or breath of the wearer and to generate an electric current and metal ions when the metal ions bind with ambient oxygen or oxygen from the skin of the wearer. The generation of such an electric current kills microbes in the vicinity. Another added advantage is that the zinc-air battery may reduce mask rash, and zinc and oxygen are healthy for the skin.

The fabric described herein also may be used in the manufacture of various personal protective equipment such as gowns, scrubs, caps, etc., as well as various clothing items as well as sheets, pillow cases, towels, wipes, etc. that may come into contact with or close proximity to the skin. FIG. 17 shows various examples of personal protective equipment made in accordance with the present invention including hospital gowns, caps, as well as sheets and pillow cases, etc.

Various changes may be made in the above invention without departing from the spirit and scope. For example, the fibers may be co-extruded to have a center or core of the same or dissimilar polymer with the metal filled polymer on the outside of the fiber. Co-extrusion has the advantage that the center of the fiber is void of metal and therefore can contribute more strength to the fiber, while the outer layer may be loaded with metal particles. Or, the metal filled polymer may be intermittently dispersed into discrete reservoirs within the fiber during fiber formation. And, of carbon fiber nanotubes (hollow-tubes) can be added to provide increased tensile strength as well as the antimicrobial nature of the hollow tubes. The carbon nanotubes also are electrically conductive and will electrically connect the zinc particles in the reservoir into a layer mass so that the available zinc ions are interconnected providing a layer capacity or discharge. Additionally, if there is a recharging effect of free floating H+ ions, then the carbon nanotubes also will enable more even recharging of the zinc mass. Also, the amount of metal particles in the fibers may be adjusted to adjust the capacity or voltage of the air battery in the thread or yarn.

Alternatively, as shown in FIG. 18 metal particles, typically metallic zinc particles which may be previously formed by grinding or precipitated out of suspension, and having an average particle size between 1 and 100 nanometers, more preferably 1-10 microns, even more preferably about 5 microns are mixed with a thermoplastic material such as polyethylene in a heated mixing vat to melt the material, and the mixture bump extruded or melt spun at spinning station to form fibers 114, having thicker portions 314A of metal particles 316 filled filaments and thinner portions 314B of metal particles 316 filled filaments therebetween (see FIG. 19). The polyethylene is the polymer of choice for releasing of electrons from the metal. The porosity of the fiber also is believed to play a part. Polyacrylic or polyester fibers also may be used however the result is a slower ion release. The metal particles filled fibers may then be cabled or twisted at a cabling station, and woven at a weaving or knitting station into a garment such gloves, hats, socks, underwear, bras and underbra inserts, shirts, leggings, tights, compression clothing or a cloth which may be made into a therapeutic wrap for use in treating hyperhidrosis, neuropathy and other condition as described in our aforesaid ‘761 and ‘172 patents, incorporated herein by reference.

And, while fabric coated with zinc particles as described above advantageously may be use in forming deposable PPEs fabric coated with elemental zinc particles as described above formed by printing zinc particles on the surface of the fabric have limited washability and abrasion resistance when used for forming multiple use items such as sheets and clothing. Also, in the case of thermoplastics, once we exceed about 30% solids in the melt, the strength of the fiber drops considerably. There are many thermosetting and thermoplastic polymers as well as other “binders” such as printer’s ink, silicone, natural collagen or cellulose binders that could be used to suspend the metal powder (or salt thereof) or combination of metals within the fiber, thread or yarn. However, prior to the present invention, no one has successfully produced metal-filled fabrics having good washability and abrasion resistance.

Accordingly, an other aspect of the present invention provides a method for producing metal-filled fabrics, i.e., fabrics having elemental zinc particles or other elemental metal particles, as well as oxides and salts of such metals or combinations of metals with other chemicals carried in or on a fabric, to fabrics so produced, and to methods for treating various conditions using the so produced fabrics.

More particularly, in one aspect the present invention provides method for producing metal particle filled fibers and to metal particle filled fibers produced thereby. In another and preferred aspect, the metal particles include zinc particles, zinc oxide particles, or zinc salt particles.

In another and preferred aspect, the metal particles have a particle sized range of 1 micron - 200 microns, more preferably 2 - 100 microns, even more preferably 2-10 microns. The metal particles preferably have an average particle size of less than about 10 microns, more preferably less than about 6 microns, even more preferably less than about 5 microns. The reason for these limitations are purely practical since the fiber spinnarettes will plug up if the particles are too large or if they clump together. In addition, if there is too much filler compared to polymer, the fiber will weaken. We could add the reinforcing carbon fiber nanotubes to increase the polymer tensile strength but doing so takes up space in the polymer that we would prefer to fill with the metal.

In still another aspect, the metal particles preferably comprise about 50 and 50 %, by volume, of the fiber, more preferably about 40 - 60 volume % of the fiber, even more preferably between about 20 -30 volume % of the fiber.

In yet another aspect of the invention, the metal particles are dispersed as micro pellets within the fiber material.

In yet another aspect, the metal particle filled fiber material is formed by dispersing metal particles throughout the fiber during fiber formation.

In yet another aspect of the invention, the metal particle containing fiber is formed by mixing the metal particles with a thermosetting setting plastic material such as a polyester resin or a vinyl ester resin and forming the mixture as elongate fibers or threads as it sets. Alternatively, the metal particles can be dusted onto the setting fibers or threads.

In yet another aspect of the invention, the metal particle containing fiber is formed by spinning, drawing or extruding a heated thermoplastic material such as a polyolefin such as polyethylene or polypropylene, a polyamide such as nylon, or an acrylic, containing the metal particles.

The amount of metal available per fiber can be manipulated to increase/decrease concentration and spacing of reservoirs of the metal within the fiber. Metal availability also may be controlled by particle size or particle size distribution. Very fine particles may become coated with binder more than larger particles. However, the binder can be manipulated to expose more of the particle to the contact area. By controlling the particle size, performance of the fiber will differ.

The amount of metal available per thread or yarn also can be manipulated to increase/decrease concentration and spacing of reservoirs of the metal within the thread or yarn. This may be done at the fiber level by adjusting the amount of metal held within the fiber and how the metal is attached to the fiber. We can fill the fiber with a large amount or a small amount of metal, or we can co-extrude metal filled fiber over another fiber so the only part of the fiber loaded with metal is the outer wrap. We also can manipulate the extrusion to create pockets of high and low metal concentrations, or no metal at all.

In the case of a monofilament we can “bump extrude” the filament with metal to produce thicker portions metal filled filament and thinner portions created by the frequency of the “bumps”.

By controlling the amount and particle size of metals in the fiber and how the metal is bound to the fiber, we can adjust slow or fast release of ions. We also can increase or decrease the reservoir capacity within the fiber and subsequently the capacity of the battery created when combined with oxygen.

Referring again to FIG. 11, according to another embodiment of the invention, metal particles, typically metallic zinc particles having an average particle size between 1 and 100 microns, preferably 1-10 microns, even more preferably about 5 microns are mixed with a thermosetting polymer material such as polyester chips in a melting vat 122. The molten mixture is expressed through a spinneret at station 124 to form an elongate thread having metal particles incorporated into the thread with the metal particles exposed at least in part on the surface of the thread. Alternatively, pure polyester chips may be spun or pulled from the melt, and dusted with metal particle as the thread sets. The thread is then cabled or twisted at a cabling station 126, woven into cloth at a weaving station 128, and the cloth formed into a textile product or wrap at step 130.

Referring to FIG. 19, an embodiment of Applicants’ device for treating hyperhidrosis is illustrated. As shown, Applicants’ device comprises an underbra insert 300 that includes a fabric 310 and a plurality of metal deposition areas 320. As shown, the plurality of individual metal deposition areas 320 are discontinuous and uniformly distributed on the surface of the fabric 310, in imaginary spaced lines or lines of dots, to cover a substantially consistent percentage of the surface area of the fabric 310. Typically, the lines or lines of dots are evenly spaced at spacings from 0.1 to 3 mm, preferably 0.2 to 2 mm, more preferably 0.3 to 1.5 mm, most preferably 0.5 to 1.0 mm. The concentration of zinc in the binder that forms the line or deposition determines the amount of zinc available for the battery. Preferred concentration is 30% but the lowest is about 1% and the highest about 50%. The mixture of binder and metal forms a paste that can be applied by silk screening wherein the paste viscosity is important. A 30% by weight zinc to binder is preferred for this. The line width and length also determines the amount of zinc in the deposition since the wider and longer the line, the more zinc is available. Preferred line or line of dots width is 1 mm width but width can vary from 0.1 mm up to 5 mm width. Since the deposition is on a fabric, the amount of binder/zinc applied also can be varied. In certain embodiments, the article being coated can be coated twice or more times over the same spot wherein the thickness of the deposition can be increased as desired. In certain embodiments, the metal deposition area patterns 320 cover from about 10% to about 90% of the surface area of the fabric. In other embodiments, the metal deposition areas 320 cover from about 1% to about 99%, from about 5% to about 95%, from about 10% to about 90%, or from about 15% to about 85%, from about 20% to about 80%, from about 30% to about 70%, from about 35% to about 65%, from about 40% to about 60%, from about 45% to about 55%, or about 50%, of the surface area of the fabric 110. Although FIG. 19 shows the plurality of metal deposition areas 320 substantially uniformly distributed on the surface of the fabric 310, in other embodiments, the plurality of metal deposition areas 320 randomly may be distributed on the surface of the fabric 310. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes as shown, for example, straight continuous lines are shown in FIG. 20A; straight broken lines are shown in FIG. 20B; continuous saw-shaped as shown in FIG. 20C; continuous wavy lines as shown in FIG. 20D; broken wavy lines as shown in FIG. 20E, etc. The actual shape of the lines is not important. Preferably, but not necessarily, the lines are approximately equal in thickness and are evenly spaced.

The underbra insert fabric 310, as illustrated in the embodiment of FIG. 19, comprises a single layer. However, in other embodiments, the fabric 310 may comprise one, two, or three or more layers of fabric including metal deposition areas on at least one surface of the device. The underbra insert 300 is worn inside a bra cup underneath the breast in contact with the skin as a bra underliner to treat excessive sweating associated with hyperhidrosis.

Preferably, but not necessarily, the fabric 310 comprises a woven textile, a non-woven textile, a fibrous mesh, a non-fibrous mesh, a textile mesh, or the like. In one embodiment, the fabric may comprise a polymeric film or a polymeric coating. In an embodiment, the fabric may be interwoven with elastic fibers, elastic bands, or metallic fibers. In certain embodiments, the fabric is electrically conductive or electrically nonconductive.

In certain embodiments, fabric 310 is permeable to ambient air. In certain embodiments, the plurality of individual metal deposition areas 320 comprise elemental zinc particles.

In one embodiment, the device includes a fastener configured to attach the device or the underbra insert 300 to the skin surface or to the surface of a cloth article. For example, referring again to FIG. 19, in certain embodiments the surface of the fabric 310 comprises a surface of the fabric 310 including the plurality of metal deposition areas 320 in contact with the skin and an opposing surface of the fabric 110 in contact with an a cloth article. In certain embodiments, the opposing surface of the fabric 110 includes an adhesive configured to attach the fabric 310 to a cloth article. For example, the underbra insert 300 as shown in FIG. 19 includes the plurality of metal deposition areas 320 on one surface of the fabric 310 configured for contact with the skin surface. An opposite surface of the underbra insert 300 (not shown) includes an adhesive or adhesive strips configured to adhere the underbra insert 300 to the interior of a bra surface. In an embodiment, the device is configured for attachment to a cloth article via at least one of the group consisting of a hook and loop fastener, buttons, zippers, electrostatics, an adhesive, a hook and eye fastener, a thread, snaps, or the like.

In an embodiment, the surface of the fabric 310 including the plurality of metal deposition areas 320 further comprises an adhesive for attachment of the fabric to the skin surface.

In an embodiment, the fabric of the device comprises a cloth article. For example, the fabric includes at least one member selected from the group consisting of a sock, a glove, a scarf, a headband, a cap, a hat, a face mask, a respirator, a t-shirt, a bra, an underarm or underbra insert, pants, sleeves, underwear (undergarment clothing in contact with the skin), or compression clothing such as ankle, arm or knee sleeves, shorts and shirts, or sheets and pillowcases, towels and drapes.

In certain embodiments, zinc is utilized as a powdered elemental crystal. In certain embodiments, the zinc utilized has a purity of about 99.99 percent however, zinc is available in other purities and particle sizes as defined by the user. In certain embodiments, the zinc comprises a -325 mesh size. As those skilled in the art will appreciate, particles passing through a -325 mesh are considered the “fines.”

In certain embodiments, the zinc particles are very uniform in size. In certain embodiments, the zinc particle size distribution is between about 4 microns to about 10 microns in diameter. These individual particle crystals approach the visible range and are easily seen as shiny crystals on the surface.

1-28. (canceled)

29. A method of protecting an individual from live airborne bacteria, fungi, molds and viruses, comprising providing the individual with a cloth article formed of a fabric having nanosize particles of zinc disposed on or exposed through said fabric as a plurality of lines or dots in a pattern that positions said particles in discrete physically isolated locations, wherein the particles of zinc and oxygen and moisture from ambient air and oxygen and moisture from exhaled air and skin of the individual form half cells of a battery which creates an electrical field that inactivates or kills live airborne bacteria, fungi, molds and viruses passing in and out through the fabric.

30. The method of claim 29, wherein the fabric comprises fibers in a pattern selected from the group consisting of a knit pattern and a weave pattern, wherein the fibers are spatially separated within the fabric to set up an electric field as determined by the weave pattern or the knit pattern.

31. The method of claim 29, wherein the fabric comprises fibers and the fiber surface area of the fabric is increased by a method selected from the group consisting of sanding, flocking, felting and terrying.

32. The method of claim 29, wherein the fabric is manufactured in a process selected from the group consisting of weaving, knitting, gluing or non-woven, wherein the fabric comprises threads or filaments having particles of zinc, threads or filaments having particles selected from the group consisting of copper, silver and magnesium, and at least one neutral insulating fiber or at least one neutral insulating thread.

33. The method of claim 29, wherein the fabric is formed of fibers or filaments having particles selected from the group consisting of zinc, zinc oxide and zinc salt, and fibers or filaments having particles selected from the group consisting of copper, silver, magnesium, copper oxide, silver oxide, magnesium oxide, a copper salt, a silver salt, and a magnesium salt, wherein the fabric is manufactured using a process selected from the group consisting of woven, knitted, glued or thermally fused, and wherein at least some of the fibers or filaments of the fabric are separated at least in part from one another.

34. The method of claim 29, wherein the fabric is formed of fibers having nanosized particles of zinc disposed on or exposed through said fabric as a plurality of lines or dots in a pattern that positions said particles in discrete physically isolated patterns, wherein the zinc particles cover about 1% to 99%, from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, or from about 50% of the surface area of the fabric.

35. The method of claim 34, wherein the fibers contain carbon nanotubes dispersed intermittently within the fibers during fiber formation, and wherein the particles of zinc are selected from the group consisting of elemental zinc particles, zinc oxide and zinc salt.

36. The method of claim 29, wherein the zinc particles have a size range of 1 to 1,000 nanometers, 1 to 500 nanometers, or 1 to 100 nanometers.

37. The method of claim 34, wherein the fibers comprise thermosetting thermoplastic fibers, preferably polyethylene fibers or polypropylene fibers.

38. The method of claim 34, wherein the fibers are formed by co-extruding polyethylene fibers with a core fiber formed of the same or a different thermoplastic material or with a thermosetting material.

39. The method of claim 29, wherein the cloth article comprises personal protective equipment, wherein a surface of the personal protective equipment is configured to be in close or direct contact with the skin of the wearer, at least in part, when worn, and wherein the particles are arranged so that the fabric in close or direct contact with the skin of the wearer forms a plurality of half-calls of an air-zinc battery.

40. The method of claim 29, wherein the cloth article is selected from the group consisting of socks, gloves, headbands, caps, scarves, face masks, respirators, face cloth coverings, hats, t-shirts, scrubs, gowns, caps, leggings, tights, underwear, underarm and under bra inserts, bras, and compression clothing, ankle sleeves, arm sleeves and knee sleeves, elastic bandages and wraps, sheets, pillowcases, towels and drapes, in which the particles of metal exposed at least in part on the surface of the polyethylene fibers contact the skin of the user.

41. The method of claim 29, wherein the fabric material comprises polyethylene fiber sections containing the particles of metal and polyethylene fiber sections devoid of particles of metal.

42. The method of claim 29, wherein the fabric material further includes a drug carried by/on the fabric material.
